(19) United States
(12) Patent Application Publication
(10) Pub. No.: US 2013/0071361 A1
(43) Pub. Date: Mar. 21, 2013

(54) METHODS OF TREATMENT OF HEMOPHILIA

(71) Applicants: Sanjeev Gupta, Scarsdale, NY (US); Antonia Follenzi, Torino (IT)

(72) Inventors: Sanjeev Gupta, Scarsdale, NY (US); Antonia Follenzi, Torino (IT)

(21) Appl. No.: 13/578,757

(22) PCT Filed: Feb. 14, 2011
(86) PCT No.: PCT/US11/00266

§ 371 Date: Oct. 16, 2012

Related U.S. Application Data
(60) Provisional application No. 61/338,514, filed on Feb. 18, 2010.

(51) Int. Cl. A61K 35/14

(52) U.S. Cl. CPC A61K 35/14
USPC 424/93.7

(57) ABSTRACT

The present invention provides methods of treating hemophilia in a subject, the methods comprising transplanting a therapeutically effective amount of bone marrow, cord blood, cord blood or bone marrow fraction expressing Factor VIII, mononuclear cells, or mesenchymal stromal cells from a donor to the subject with hemophilia. The present invention also provides compositions for treating hemophilia.

### CROSS-REFERENCE TO RELATED APPLICATIONS

This application claims benefit of U.S. Provisional Application No. 61/338,514, filed Feb. 18, 2010, the contents of which are hereby incorporated by reference.

### STATEMENT OF GOVERNMENT SUPPORT

The invention disclosed herein was made with government support under grant numbers R01 DK071111, P30 DK41296, and P30 CA13330 awarded by the National Institutes of Health, U.S. Department of Health and Human Services. Accordingly, the government has certain rights in the invention.

### FIELD OF THE INVENTION

The present invention relates generally to methods of treating hemophilia in a subject.

### BACKGROUND OF THE INVENTION

Throughout this application various publications are referred to in parenthesis. Full citations for these references may be found at the end of the specification. The disclosures of these publications are hereby incorporated by reference in their entirety into the subject application to more fully describe the art to which the subject invention pertains.

The bleeding disorder, hemophilia A, arises from mutations in FVIII gene and is transmitted in an X-linked manner, with 1 to 2 cases per 10,000 males (1). Currently, hemophilia A cannot be cured. To prevent major bleeding episodes in hemophilia, hFVIII protein is frequently administered. This is complicated by its high cost and development of antibodies that neutralize FVIII activity in 20 to 30% of patients. Therefore, permanent solutions in the form of cell and gene therapy are very attractive for hemophilia A. Since relatively small amounts of FVIII may substantially decrease bleeding risk in hemophilia, limited replacement of healthy cells producing FVIII will be sufficient. However, this needs insights into cell types capable of efficiently synthesizing and releasing FVIII, especially in proximity to von Willebrand factor, which protects FVIII from premature degradation (2). Although the cell type-origin of FVIII has been controversial, in part due to the lack of suitable FVIII antibodies to localize this protein in cells, orthotopic liver transplantation (OLT) rapidly corrected hemophilia, suggesting a major role of liver in FVIII production (3, 4). Subsequently, cell transplantation studies established that liver sinusoidal endothelial cells (“LSEC”) were largely responsible for hepatic FVIII production (5, 6). Endothelial cells in other organs likely produce FVIII, as indicated by FVIII synthesis and release from spleen, lungs, or pancreatic islet endothelial cells (5, 7, 8). Moreover, absence of FVIII deficiency after OLT with donor liver from a person with hemophilia A, suggested that FVIII may be produced in extrahepatic locations (9). However, most experts now agree that donor bone marrow (BM)-derived mature hepatocytes or LSEC represent rare events, despite the proposed existence of a shared hematopoietic and endothelial stem cell, the hemangioblast (12, 13). Since achievement of therapeutic cures in haemophilia A required replacement of over 10% of the total liver sinusoidal endothelial cell (LSEC) compartment, rare endothelial cells generated by donor BM will be insufficient for curing haemophilia A (6). Therefore, therapeutic correction should require significant replacement of FVIII-producing cells of other types.

The present invention provides a therapeutic cure for hemophilia by introduction of Factor VIII-producing cells into the subject.

### SUMMARY OF THE INVENTION

A method of treating a subject with hemophilia, the method comprising introducing into the subject a therapeutically effective amount of Factor VIII-producing cells so as to thereby treat the subject with hemophilia.

A composition, or pharmaceutical composition, for treatment of hemophilia comprising an amount of Factor VIII-producing mononuclear cells or Factor VIII-producing mesenchymal stromal cells and a pharmaceutically acceptable carrier.

A composition, or pharmaceutical composition, for treatment of hemophilia comprising an amount of (a) (i) Factor VIII-producing bone marrow or Factor VIII-producing bone marrow fraction, or (ii) Factor VIII-producing cord blood cells or Factor VIII-producing cord blood fraction; or (i) and (ii); and (b) a pharmaceutically acceptable carrier.

Use of Factor VIII-producing bone marrow or Factor VIII-producing bone marrow fraction from a donor subject for the treatment of hemophilia. Use of Factor VIII-producing cord blood cells or Factor VIII-producing cord blood fraction from a donor subject for the treatment of hemophilia. Use Factor VIII-producing mononuclear cells or Factor VIII-producing mesenchymal stromal cells from a donor subject for the treatment of hemophilia. In an embodiment the hemophilia is hemophilia A.

The present invention provides a method of treating a subject with hemophilia, the method comprising transplanting a therapeutically effective amount of bone marrow from a donor to the subject with hemophilia. The present invention further provides a method of treating a subject with hemophilia, the method comprising introducing a therapeutically effective amount of cord blood from a donor to the subject with hemophilia. The present invention additionally provides a method of treating a subject with hemophilia, the method comprising introducing a therapeutically effective amount of a composition comprising a cord blood fraction or a bone marrow fraction which expresses Factor VIII from a donor to the subject with hemophilia.

The present invention also provides a method of treating a subject with hemophilia, the method comprising introducing a therapeutically effective amount of a composition comprising mononuclear cells or mesenchymal stromal cells from a donor to the subject with hemophilia.

The present invention provides a composition for treatment of hemophilia comprising mononuclear cells or mesenchymal stromal cells in a pharmaceutically acceptable carrier. The present invention also provides a composition for treatment of hemophilia comprising bone marrow in a pharmaceutically acceptable carrier. The present invention further provides a composition for treatment of hemophilia comprising cord blood cells in a pharmaceutically acceptable carrier. The present invention additionally provides a composition for treatment of hemophilia comprising a cord blood fraction or bone marrow fraction which expresses Factor VIII in a pharmaceutically acceptable carrier.

The present invention provides for the use of bone marrow from a donor for the treatment of hemophilia. The present invention further provides for the use of cord blood cells from a donor for the treatment of hemophilia. The present invention additionally provides for the use of a cord blood fraction or bone marrow fraction which expresses Factor VIII from a donor for the treatment of hemophilia. The present invention also provides for the use of mononuclear cells from a donor of the treatment of hemophilia. The present invention additionally provides for the use of mesenchymal stromal cells from a donor for the treatment of hemophilia.

### BRIEF DESCRIPTION OF THE DRAWINGS

FIG. 1A-1G. Replacement of bone marrow (“BM”) cells in hemophilia A mice. (1A) Experimental timeline indicating animal groups subjected to BM transplantation (Tx), including no further treatment (NT) or J0-2 antibody, MCT, and CCl₄ followed by assays for therapeutic correction. (1B) Flow cytometry showing BM chimerism in recipients of Rosa 26 BM with LacZ expression detected by fluorescein di[β-D-galactopyranoside](FDG). Panels on left indicate negative controls. R3 gate in panels on right indicates transplanted BM-derived cells. BM chimerism was extensive, including in mice treated after BM Tx with J0-2, MCT and CCl₄. (1C) Flow cytometry in a recipient of GFP transgenic BM cells showing chimerism of 90% in blood and 60% in BM. (1D) LacZ staining showing its absence in hemophilia A mouse without BM transplant. (1E) Donor BM-derived Rosa26 cells identified by LacZ immunostaining (arrows) in sinusoids 3 months after transplant. (1F, 1G) GFP transgenic BM-derived cells in mice 6 months or 1 year after transplants, again with transplanted cells (arrows) in liver sinusoids. Original magnification, E-H, x400.

FIG. 2A-2b. Correction of hemophilia A after transplantation of healthy BM. (2A) Factor VIII (“FVIII”) expression in BM was assessed by RT-PCR of total RNA from: lanes 1-13, BM-recipient hemophilia A mice; lane marked (+), BM from GFP transgenic donor mouse; lane marked (−), hemophilia A mouse liver; lane marked (W), PCR mix alone. Expression of β-actin indicates RNA integrity. (2B) Therapeutic correction in BM transplanted hemophilia A mice, as well as subgroup analysis in recipients of 2×10⁶ and 10×10⁶ BM cells from the first group of 86 surviving mice with no further treatment.

FIG. 3A-3C. Therapeutic efficacy of BM transplant in hemophilia A. (3A, 3B) Plasma FVIII activity in mice treated with 2×10⁶ or 10×10⁶ BM cells from GFP transgenic donors. The data were obtained 4 to 6 months after BM transplantation. (3B) Plasma FVIII antigen levels in hemophilia A mice 2, 4 and 6 months after BM transplantation. (3C) Correlation between plasma FVIII antigen levels and FVIII activity in mice 4 and 6 months after BM transplantation, where both sets of data were available for comparison.

FIG. 4A-4B. Tail-clip assay in hemophilia A mice with BM transplantation. (4A) Shows survival after tail-clip of mice −8, 12, 30 and 52 weeks after BM transplantation. The number of total mice in each group is given at the bottom of the bars and number of surviving mice and fractional survival of animals is shown at the top of the bars. (4B) Analysis of FVIII activity in mice surviving and not surviving after tail-clip assay.

FIG. 5A-5E. FVIII expression in BM and cord blood (“CB”) cells. (5A) FVIII expression in human BM cells by RT-PCR of total RNA: lane 1, molecular weight marker; lane 2, CD105 (mesenchymal and vasculogenic endothelial) cells; lane 3, CD33 (myeloid) cells; lane 4, mesenchymal stromal cells; lane 5, CD133 (hematopoietic precursor) cells; lane 6, total BM mononuclear cells (MNC); lane 7, adult human hepatocytes (Hep); lane 8, fetal human liver (FL); and lane 9, PCR mix (W) alone. β-actin was simultaneously amplified to verify RNA integrity. (5B) RT-PCR of total RNA for FVIII in CD34+ human CB cells (lane 1), total human CB cells (lane 2), and BM-derived hMSC used for transplantation studies in NOD/SCID hemophilia A mice (lane 3). Lane 4 shows negative control with PCR mix alone (W). (5C) Flow cytometry showing analysis of BM from hemophilia A mouse, GFP transgenic donor mouse and hemophilia mice 9 months after transplantation of BM from GFP donor mouse. Notice replacement of CD105+ BM cells in BM recipients. (5D, 5E) Showing FVIII immunostaining of BM-derived hMSC in culture with absence of any signals when FVIII antibody was omitted (5D) and identification of FVIII protein in the cytoplasm after staining with FVIII antibody (5E).

FIG. 6. DNA PCR analysis showing donor-derived BM cells in multiple tissues. Lane 1, molecular weight (MW) marker; lane 2, Rosa26 donor; lane 3, nontransplanted hemophilia A mouse; lanes 4-8, liver, heart, spleen, lung and BM from a hemophilia A recipient of Rosa26 BM. The bands shown are for LacZ transgene and endogenous c-mos gene.

FIG. 7A-7B. Nature of liver cells generated by donor-derived BM cells 3-6 months after BM transplantation. (7A) Shows typical flow cytometric profiles of GFP transgenic BM-derived cells with gating of total nonparenchymal liver cells (NPC), GFP and CD146-positive LSEC or GFP−+ hepatocytes (R4 regions in panels at top). Cells were isolated by collagenase digestion of recipient liver. Bottom panels show fractions in NPC of CD45−+ blood cells and CD11b−+ monocytes/macrophages, which were the majority, with very few other cells (1% or less). (7B) FVIII mRNA in fractionated cells. This indicated FVIII expression in CD11b+, CD45+ and LSEC from one of the two representative mice shown. Contamination of the LSEC fraction with mononuclear cells was not excluded. Expression of b-actin shows RNA integrity. The requirement of nested PCR (2 PCR rounds) indicated extremely low levels of FVIII mRNA, which was different from abundance of FVIII mRNA in mature LSEC; therefore, this was in agreement with the absence of BM-derived LSEC.

FIG. 8. Expression of FVIII mRNA in Kupffer cells (KC). Shows RT-PCR of 1 μg total RNA for FVIII expression in CD11b+ KC isolated from hemophilia A and healthy C57BL/6 mice by liver perfusion. Cells were cultured for 48 hours to remove nonadherent cells and RNA was isolated by Trizol. Lane 1, KC from hemophilia A mouse; Lane 2, KC from C57BL/6 mouse; Lane 3, PCR mix alone. Expression of β-actin shows RNA integrity. KC in culture were found to express FVIII protein by immunostaining studies (not shown).

### DETAILED DESCRIPTION OF THE INVENTION

A method of treating a subject with hemophilia, the method comprising introducing into the subject a therapeutically effective amount of Factor VIII-producing cells so as to thereby treat the subject with hemophilia.

In an embodiment the Factor VIII-producing cells are (i) bone marrow cells or (ii) a bone marrow cell fraction, obtained from a donor subject. In an embodiment the Factor VIII-producing cells are introduced into the subject with hemophilia by transplantation. In an embodiment the Factor VIII-producing cells are cord blood cells, or a cord blood cell fraction, or mobilized peripheral blood cells. In an embodiment the cord blood is introduced intravenously into the subject with hemophilia. In an embodiment the Factor VIII-producing cells comprise CD34+ cells. In an embodiment the Factor VIII-producing cells comprise CD133+ cells. In an embodiment the Factor VIII-producing cells comprise mononuclear cells. In an embodiment the Factor VIII-producing cells comprise mesenchymal stromal cells. In an embodiment the mononuclear cells comprise macrophages. In an embodiment the macrophages are Kupffer cells. In an embodiment the mononuclear cells or mesenchymal stromal cells are extracted from bone marrow of the donor subject. In an embodiment the mononuclear cells or mesenchymal stromal cells are extracted from peripheral blood of the donor subject. In an embodiment the mononuclear cells are expanded in vivo before extraction. In an embodiment the mesenchymal stromal cells are cultured in vitro after extraction from the donor subject. In an embodiment the mononuclear cells or mesenchymal stromal cells are derived from stem cells of the donor. In an embodiment the stem cells of the donor are not embryonic stem cells. In an embodiment the subject with hemophilia and the donor subject are both mammals. In an embodiment the subject with hemophilia and the donor subject are both human. In an embodiment the donor subject and the subject with hemophilia are the same subject in that the cord blood was previously obtained from the umbilical cord of the subject when the subject was neonatal. In an embodiment the Factor VIII-producing cells are introduced parenterally. In an embodiment the Factor VIII-producing cells are introduced by transfusion. In an embodiment the Factor VIII-producing cells are non-megakaryocytic, nonendoithelial cells. In an embodiment the cells are transplanted into the cavity of a bone of the subject with hemophilia. In an embodiment the subject with hemophilia is a subject with hemophilia A.

A composition for treatment of hemophilia comprising an amount of Factor VIII-producing mononuclear cells or Factor VIII-producing mesenchymal stromal cells and a pharmaceutically acceptable carrier.

A composition for treatment of hemophilia comprising an amount of (a) (i) Factor VIII-producing bone marrow or Factor VIII-producing bone marrow fraction, or (ii) Factor VIII-producing cord blood cells or Factor VIII-producing cord blood fraction; or (i) and (ii); and (b) a pharmaceutically acceptable carrier.

Use of Factor VIII-producing bone marrow or Factor VIII-producing bone marrow fraction from a donor subject for the treatment of hemophilia. Use of Factor VIII-producing cord blood cells or Factor VIII-producing cord blood fraction from a donor subject for the treatment of hemophilia. Use Factor VIII-producing mononuclear cells or Factor VIII-producing mesenchymal stromal cells from a donor subject for the treatment of hemophilia. In an embodiment the hemophilia is hemophilia A.

Hemophilia is a group of hereditary genetic disorders that impair the body's ability to control blood clotting or coagulation mechanisms normally used to stop bleeding when a blood vessel is broken. Hemophilia A, the most common form of hemophilia, is a deficiency of clotting factor VIII (“Factor VIII”) production in a subject. Hemophilia B is due to a deficiency in clotting factor IX. Hemophilia C is due to a deficiency in clotting factor XI. Many different mutations are responsible for each type of hemophilia. The mutations may result in absent, nonfunctional, or depressed levels of the protein. The deficiency in the clotting factor prevents the formation of fibrin, necessary to maintain a blood clot after injury, in hemophiliacs. Depending on the severity of hemophilia, a small injury may result in bleeding lasting for abnormal and long periods. Additionally, such bleeding may be fatal.

Transfusion of bone marrow, or introduction of cord blood cells, cord blood fractions or bone marrow fractions expressing Factor VIII, mononuclear cells or mesenchymal stromal cells, from a donor with the ability to replace the clotting factor will treat the hemophiliac. Since hemophiliacs suffering from hemophilia B produce clotting factors VIII and XI, their bone marrow, mononuclear cells, or mesenchymal cells can be used to treat hemophilia A or C. Ideally, the donor will not be suffering from any form of hemophilia.

“Bone marrow” as used herein is the spongy flexible tissue found in the hollow interior of many mammalian bones. There are two types of bone marrow: red marrow (consisting mainly of hematopoetic tissue) and yellow marrow (consisting mainly of fat cells). Red blood cells, platelets and most white blood cells arise in red marrow. Both types of bone marrow contain numerous blood vessels and capillaries. The stroma of the bone marrow is all tissue not directly involved in the primary function of hematopoiesis. The yellow bone marrow belongs here, and makes the majority of the bone marrow stroma, in addition to stromal cells located in the red bone marrow. Still, the stroma is indirectly involved in hematopoiesis, since it provides the hematopoietic microenvironment that facilitates hematopoiesis by the parenchymal cells. Cells that constitute the bone marrow stroma are: fibroblasts (reticular connective tissue), macrophages, adipocytes, osteoblasts, osteoclasts, and endothelial cells forming the sinusoids. Macrophages contribute especially to red blood cell production and deliver iron for hemoglobin-production. Bone marrow contains three types of stem cells: hematopoietic stem cells give rise to the three classes of blood cells that are found in the circulation—white blood cells (leukocytes), red blood cells (erythrocytes), and platelets (thrombocytes); mesenchymal stem cells are found arrayed around the central sinus in the bone marrow and have the capability to differentiate into osteoblasts, chondrocytes, myocytes, and many other types of cells as well as function as “gatekeeper” cells of the bone marrow; endothelial stem cells.

“Cord blood” is blood that remains in the placenta and in the attached umbilical cord after childbirth. Cord blood is obtained from the umbilical cord around the time of childbirth, after the cord has been detached from the newborn. Cord blood contains stem cells, including hematopoietic cells, which can be used to treat hematopoietic and genetic disorders. Cord blood can be used to regenerate bone marrow.

Cord blood and bone marrow can be fractioned, or separated, into different fractions. Fractions which express Factor VIII can be used to treat hemophilia. Examples of cord blood fractions and bone marrow fractions which express Factor VIII include, but are not limited to, CD34+ and CD133.

As used herein a “mononuclear cell” is a specialized blood cell having a round nucleus including, for example, monocytes, and macrophages. Mononuclear cells are a critical component in the immune system to fight infection and adapt to intruders. Mononuclear cells are derived from myeloid progenitor cells. By contrast, the lymphocyte population consists of T cells (CD4 and CD8 positive ˜75%), B cells and NK cells (˜25% combined). The monocyte population consists of white blood cells that can divide to replenish macrophages and dendritic cells as well as respond to inflammatory signals and includes the “classic” CD14++ monocyte and the pro-inflammatory CD14+CD16+ monocyte. The macrophage population consists of white blood cells produced by the division of monocytes and include, for example, Kupffer cells, sinusoidal lining cells residing in the liver. Mononuclear cells can be found in bone marrow and are also a component of the peripheral blood. Mononuclear cells can be isolated from bone marrow or whole blood. If isolating mononuclear cells from a donor's whole blood, it is preferable to expand the number of mononuclear cells in the donor's blood before harvesting blood. This can be done by any method known in the art including, but not limited to, administering granulocyte macrophage stimulating factor (“GMCSF”) to the donor before harvesting blood. Expanding the number of circulating mononuclear cells in the donor before drawing blood increases the yield of donor mononuclear cells.

As used herein, “mesenchymal stromal cells” (“MSCs”) are multipotent stem cells that can differentiate into a variety of cell types. Cell types that MSCs have been shown to differentiate into in vitro or in vivo include osteoblasts, chondrocytes and adipocytes and which can be isolated from a variety of tissues, such as bone marrow, periosteum, trabecular bone, adipose tissue, synovium, skeletal muscle, dental pulp. Additionally, MSCs can be isolated from whole blood. MSCs removed from a donor can be expanded in vitro by any method known in the art prior to transplantation.

Transplantation can be allogenic (from donor to recipient of same species) or syngeneic, or autologous (donor and recipient are the same subject). In the present invention, the recipient is the subject with hemophilia and allogenic transplantation can be used to effect treatment of hemophilia, or autologous transplantation can be used (in that cord blood can be taken from the neonatal subject and a whole or fraction thereof administered to the subject at a later date). If donor and recipient are compatible, these infused cells will then travel to the bone marrow and initiate blood cell production. Before transplantation of bone marrow or, a portion of, or all of, the recipient's marrow can be destroyed by any method known in the art, such as with drugs, chemotherapy or radiation, and the new bone marrow is then introduced. In a preferred embodiment, only a portion of the recipient's bone marrow is destroyed and the donor's bone marrow is transplanted. Bone marrow can be harvested from the donor by any method known in the art including, but not limited to, harvesting red marrow from the crest of the ilium bone. Harvested marrow can be prepared for transplantation by any method known in the art.

Cord blood is harvested from the umbilical cord of humans at childbirth. It can be introduced in to a subject by any method known in the art, including, but not limited to, intravenous introduction.

Cord blood or bone marrow fractions which express Factor VIII can be separated from the whole cord blood or bone marrow by any method known in the art, including, but not limited to, flow cytometry, centrifugation, or affinity binding.

Mononuclear cells or mesenchymal stromal cells can also be isolated from the donor's blood by any method known in the art, including but not limited to inserting an intravenous catheter into the donor's vasculature and filtering mononuclear cells and mesenchymal stromal cells out of the whole blood. Mononuclear cells or mesenchymal stromal cells can be transfused into the recipient by any method known in the art, including but not limited to inserting an intravenous catheter into the receipient's vasculature and transfusing the cells. In another embodiment, the donor's mononuclear cells or mesenchymal stromal cells can be introduced into the abdominal cavity or an organ of the recipient. To maintain the treatment of hemophilia long-term, the transplantation of mononuclear cells or mesenchymal stromal cells via parenteral administration such as by transfusion or injection into the abdominal cavity may have to be periodically repeated. Mononuclear cells or mesenchymal stromal cells can also be derived from the donor's stem cells. Any technique known in the art can be used to derive mononuclear cells or mesenchymal stromal cells from the donor's stem cells. The donor's stem cells may be embryonic stem cells. Additionally, mononuclear cells or mesenchymal stromal cells can be derived from the donor's cord blood. Any method known in the art can be used to derive mononuclear cells or mesenchymal stromal cells from the donor's cord blood. Such methods include, but are not limited to, flow cytometry, centrifugation, or affinity binding. The mononuclear cells or mesenchymal stromal cells derived from the donor's stem cells or cord blood cells can be expanded before introduction into the subject.

Cord blood fractions or bone marrow fractions expressing Factor VIII, mononuclear cells or mesenchymal stromal cells can be introduced by any method known in the art including, but not limited to, parenteral administration. The cells may be introduced parenterally into the vascular system or abdominal cavity. When treatment is effected by parenteral administration of mononuclear cells or mesenchymal stromal cells, as opposed to a bone marrow transplant, the treatment may have to be periodically repeated. Although the effects may be long-lasting, especially when introduced into the abdominal cavity, additional introductions of cells may be needed to maintain the number of cells producing the missing clotting factor.

The mononuclear cells or mesenchymal stromal cells may be associated with other cells or a pharmaceutically acceptable carrier known in the art. In the case of parenteral administration, acceptable pharmaceutical carriers may include saline and water, among others and may be brought into association with a carrier or diluent as a suspension or solution. Pharmaceutically acceptable carriers for intravenous introduction or transfusion are known in the art.

The donor and the subject with hemophilia are preferably carefully matched to ensure immunological compatibility of the transfused bone marrow or cells with the subject with hemophilia. Transfusion of blood or bone marrow containing immunoreactive lymphocytes may result in graft-versus-host disease in the subject receiving the blood or bone marrow.

The bone marrow, mononuclear cells, or mesenchymal stromal cells can be used to treat hemophilia and are provided in amounts effective to treat hemophilia. The amounts may be readily determined by one of skill in the art. In the present invention, the bone marrow is transfused or the composition containing mononuclear cells or mesenchymal stromal cells are introduced to a subject with hemophilia in an amount and manner effective to treat the subject's hemophilia. “Treating” a subject's hemophilia, as used herein, means curing, ameliorating, or minimizing the clinical impairment or one or more symptoms of the subject's hemophilia. “Effective” to treat as used herein means effective to ameliorate or minimize the clinical impairment or one or more symptoms of the subject's hemophilia. The amount of bone marrow, mesenchymal cells, or mononuclear stromal cells effective to treat hemophilia will vary depending on the clinical severity of the hemophilia and the type of cells used. Appropriate amounts of bone marrow, mononuclear cells or mesenchymal stromal cells can be readily determined by the skilled artisan without undue experimentation.

In an embodiment, from 1×10⁸ to 10×10⁸ cells kg/subject's body weight are administered to, or transplanted into, the subject with hemophilia. In an embodiment, from 2×10⁸ to 8×10⁸ cells kg/subject's body weight are administered to, or transplanted into, the subject with hemophilia. In an embodiment, from 3×10⁸ to 6×10⁸ cells kg/subject's body weight are administered to, or transplanted into, the subject with hemophilia. In an embodiment, from 4.5×10⁸ to 5.5×10⁸ cells kg/subject's body weight are administered to, or transplanted into, the subject with hemophilia. In an embodiment, about 5×10⁸ cells kg/subject's body weight are administered to, or transplanted into, the subject with hemophilia. In embodiment the cells are human bone marrow cells, mononuclear cells, mesenchymal stromal cells, or Kupffer cells.

In an embodiment the subject is not suffering from aplastic anemia. In an embodiment the subject is not also administered recombinant FVIII.

The present invention provides a composition for treatment of hemophilia comprising mononuclear cells or mesenchymal stromal cells in a pharmaceutically acceptable carrier. The present invention also provides a composition for treatment of hemophilia comprising bone marrow in a pharmaceutically acceptable carrier. Bone marrow is a flexible tissue consisting of various cell types. The composition for treatment of hemophilia comprising bone marrow may contain mononuclear cells or mesenchymal stromal cells. The present invention further provides a composition for treatment of hemophilia comprising cord blood cells in a pharmaceutically acceptable carrier. The present invention additionally provides a composition for treatment of hemophilia comprising a cord blood fraction or stem cell fraction which expresses Factor VIII in a pharmaceutically acceptable carrier.

The present invention provides for the use of bone marrow from a donor for the treatment of hemophilia. The present invention further provides for the use of cord blood cells from a donor for the treatment of hemophilia. The present invention additionally provides for the use of a cord blood fraction or stem cell fraction which expresses Factor VIII from a donor for the treatment of hemophilia. The present invention also provides for the use of mononuclear cells from a donor of the treatment of hemophilia. The present invention additionally provides for the use of mesenchymal stromal cells from a donor for the treatment of hemophilia. Since there are three different forms of hemophilia, the donor can be suffering from hemophilia as long as the form of hemophilia suffered by the donor is different from the form of hemophilia sought to be treated. Preferably, the donor is not suffering from any form of hemophilia.

### Experimental Details

### 1. Methods and Materials

Animals and procedures. The Animal Care and Use Committee of Albert Einstein College of Medicine approved studies. BM donor mice were C57BL/6-Gt(ROSA)26Sor/J, C57BL/6-Tg(ACTbEGFP)1Osb/J, CD45 congenic B6.SJL-PTPRCPEP/BOY mice (which express CD45.1) in C57BL/6 background, and TgN(Tie2GFP)287Sato/J transgenic mice in FVB/NJ background (Jackson Labs., Bar Harbor, Me.). BM was harvested from donors by flushing femurs and tibias with DMEM-5% fetal bovine serum (FBS). BM was triturated using with 18-gauge needle and passed through 70 μm nylon mesh (Becton Dickinson, Franklin Lakes, N.J.) to obtain single cell suspension in DMEM-5% FBS. RBC were lysed in 150 mM NH₄ Cl, 10 mM NaHCO₃ and 1 mM EDTA for 10 min on ice and 2−10×10⁶ cells were injected via tail vein in 300 μl serum-free IMDM.

BM recipient C57BL/6 and FVB/N mice were from National Cancer Institute (Bethesda, Md.). Hemophilia A knockout mice carried a neomycin gene cassette in exon 16 of FVIII gene, as previously described (14). Hemophilia A mice were 8-10 weeks of age with equal numbers of males and females. These mice were given 6 Gy total body radiation twice to total 12 Gy. Although 4 doses of 1 ml/kg CCl₄ alone at 10 day intervals were planned, all mice in this group died after 3 doses of CCl₄, and this manipulation was not pursued further.

NOD/SCID hemophilia A mice were sublethally irradiated to 3.5 Gy total and BM was transplanted 24 hours after radiation by tail vein injection in 300 μl serum-free IMDM.

Cell isolation and characterization. Liver of BM-recipient mice was perfused via portal vein with buffer as previously described and cells were passed through dacron mesh with 80 μm pores and centrifuged twice under 50×g for 5 minutes to remove hepatocytes. Nonparenchymal cells (NPC) in supernatant were washed and pelleted under 350×g for 10 minutes (38). RBC were lysed for 6 minutes on ice. Kupffer cells were selected by anti-CD11b antibody, remaining hematopoietic cells were selected with anti-CD45 antibody, and LSEC were recovered with anti-LSEC antibody by immunomagnetic sorting (Miltenyi Biotec, Auburn, Calif.). For this, 2×10⁷ cells were resuspended in 200 μl Separation Buffer (phosphate buffered saline, pH 7.2, 2 mM EDTA, 0.5% bovine serum albumin, Sigma) with 20 μl per 1×10⁷ cells of anti CD11b− conjugated magnetic beads (Miltenyi Biotec, Auburn, Calif.) for 20 minutes at 4° C. Cells were pelleted under 350×g for 8 minutes at 4° C., resuspended in 500 μl Separation Buffer and applied to MS Separation Columns. CD11b− negative cells were collected for a round of positive selection using anti-CD45-conjugated magnetic beads (Miltenyi Biotec, Auburn, Calif.) for 20 minutes at 4° C. Cells were pelleted, resuspended in 500 μl Separation Buffer and applied again to MS Separation Column (Miltenyi Biotec, Auburn, Calif.). CD45-negative cells were collected for a round of positive selection with anti-LSEC magnetic particles (Miltenyi Biotec, Auburn, Calif.) for 30 minutes at 4° C. Cell separations were verified by immunostaining with APC-labeled anti-mouse CD31, anti-mouse PE-CD45 and CD11b (1 μl per 10⁶ cells; Pharmingen BD Biosciences, Franklin Lakes, N.J.) followed by flow cytometry in FACSCalibur with CellQuest software (BD Biosciences, Franklin Lakes, N.J.). Isolated cell fractions were used for RNA extraction.

Kupffer cells were isolated by collagenase liver perfusion from C57/BL6 Tg-GFP mice as described above and were separated by immunomagnetic beads for CD11b antigen (Miltenyi Biotec, Auburn, Calif.). 2×10⁶ Kupffer cells were resuspended in 300 μl of serum free IMDM and injected via tail vein into C57B1/6 hemophilia A mice (n=3) pretreated 24 hours earlier i.p. with 10 mg/kg GdCl₃ (Sigma, St. Louis, Mo.).

Analysis of BM reconstitution. Mice were assessed 8-12 weeks after BM transplantation. Flow cytometry was performed of peripheral blood and BM with phycoerythrin (PE)-conjugated anti-mouse antibody for CD45.1 (Pharmingen BD Biosciences), staining with fluorescein di[β-D-galactopyranoside](FDG, Sigma Chemical Co., St. Louis, Mo.) for LacZ, or by native GFP fluorescence in cells and tissues. Engraftment of human cells in NOD/SCID hemophilia A mice was assessed by flow cytometry with allophycocyanin (APC)-labeled anti-human CD45 (Pharmingen BD Biosciences, Franklin Lakes, N.J.).

Treatments of mice after BM transplant. Liver injury was induced by 200 μg/kg i.p. J0-2 antibody, a Fas agonist (Pharmingen BD Biosciences, Franklin Lakes, N.J.) (14), with 4 weekly injections in normal saline; 200-mg/kg i.p. MCT (Sigma, St. Louis, Mo.) in saline, plus two i.p. injections 10 days apart of 0.5 ml/kg CCl₄ diluted 1:1 (volume/volume) in olive oil; or 4 weekly i.p. injections of 1 ml/kg CCl₄.

Tissue analysis after BM reconstitution. To identify LacZ-positive cells, liver was fixed in formalin and paraffin-embedded. Sections were deparaffined, endogenous peroxidase was quenched with 3% H₂O₂, antigen was retrieved in citrate buffer, followed by blocking with 5% goat serum and 2% BSA in phosphate buffered saline (PBS). Sections were incubated in rabbit anti β-gal IgG (1:5000, Rockland Immunochemicals Inc., Gilbertsville, Pa.), followed by biotin-conjugated goat anti-rabbit antibody (1:500), and color was developed with diaminobenzidine (DAB+ kit, DAKO Cytomation) and counterstaining was with hematoxylin.

To identify GFP-expressing LSEC, liver samples were collected up to 1 year after cell transplantation and fixed in 4% PFA and frozen to −80° C. after covering in optimal cooling temperature (OCT resin). Cryostat sections of 5-μm thickness were postfixed with paraformaldehyde blocked in 5% goat serum (Vector Laboratories, Burlingame, Calif.) in PBS containing 0.1% Triton X-100 (PBS-T) for 1 hour at room temperature (RT), followed by incubation with rabbit anti-GFP in PBS-T (1:300, Molecular Probes, Life Technologies, Carlsbad, Calif.) for 1 hour at RT. After PBS washing, sections were incubated with Alexa Fluor®488-conjugated goat anti-rabbit IgG (1:500, Molecular Probes, Life Technologies, Carlsbad, Calif.) for 1 hour at RT.

To distinguish between Kupffer, endothelial and transplanted GFP-expressing cells, 5 μm-thick cryostat liver sections were blocked in 5% goat serum, 1% BSA, 0.1% PBS-T, and incubated with rabbit anti-GFP (1:300, Molecular Probes, Life Technologies, Carlsbad, Calif.), and with rat anti-mouse F4/80 (1:500 dilution, Serotec, Raleigh, N.C.) and with rat anti-mouse CD31(1:100 dilution, Pharmingen BD Biosciences, Franklin Lakes, N.J.). Sections were washed and incubated with Alexa Fluor®488-conjugated goat anti-rabbit IgG and with Alexa Fluor®546-conjugated goat anti-rat IgG (Molecular Probes, Life Technologies, Carlsbad, Calif.). CD105-stainings were performed in BM from hemophilia A, GFP transgenic and BM transplanted mice. For cytostaining, 2 μl of phycoerythrin (PE)-conjugated anti-mouse CD105 was used per 10⁶ cells (R&D Systems, Minneapolis, Minn.).

Human cells. RNA from total BM and BM fractions was commercially available (STEMCELL Technologies, Vancouver, BC, Canada). Cord blood cells were from New York Blood Center. The identity of donors was not available to investigators. Studies with human cells were approved by the Committee on Clinical Investigations at Albert Einstein College of Medicine. For transplantation, 15×10⁶ BM cells or 20×10⁶ CB cells were injected via tail vein into 12 NOD/SCID hemophilia mice each. In 12 more NOD/SCID hemophilia mice, 1×10⁵ CD34+ human CB cells were transplanted after overnight culture in medium. Some of the mice injected with CD34+ cells also received 4×10⁵ human BM-derived MSC after 3 passages in cell culture. These primary human BM-derived MSC were available commercially and were maintained in culture at 37° C. in 5% CO₂ condition for up to 7 passages in Mesencult MSC Basal Medium (human) supplemented with human Mesenchymal Stem Cell Stimulatory Supplements (STEMCELL Technologies, Vancouver, BC, Canada). For transplantation, 5×10⁶ cultured hMSC were mixed with Cytodex3 microcarrier beads (Pharmacia Amersham, GE Healthcare, Piscataway, N.J.) and were injected into the peritoneal cavity in NOD/SCID hemophilia A mice (n=3), as described previously (5). Mice were subjected to tail-clip challenge after 3 days.

DNA PCR. Genomic DNA was extracted with DNeasy Tissue Kit (Qiagen Inc., Valenica, Calif.). LacZ primers were 5′ttccgtcatagcgataacgag3′(SEQ ID NO:1) (forward) and 5′accgcatcagcaagtgtatct3′ (SEQ ID NO:2) (reverse). Platinum PCR Supermix (Invitrogen, Life Technologies, Carlsbad, Calif.) was used with 30 cycles at 94° C.×3 minutes, 94° C.×30 seconds, 55° C.×1 minutes, 72° C.×1 minute, and final elongation at 72° C.×7 minutes. Primers for mouse GAPDH were 5′gggtggagccaaacgggtc3′ (SEQ ID NO:3) (forward) and 5′ggagttgctgttgaagtcgca3′ (SEQ ID NO:4) (reverse) with 25 cycles at 94° C.×3 minutes, 94° C.×30 seconds, 56° C.×1 minutes, 72° C.×1 minute, and 72° C.×7 minutes. The products were resolved in 1% agarose gels with expected sizes of 572 bp (LacZ) and 550 bp (GAPDH). PCR was performed on multiple tissues, including liver, heart, lungs, spleen and BM.

RT-PCR assays. Total RNA was extracted from liver, BM and isolated cell population (LSEC, hepatocytes and Kupffer cells) with Trizol Reagent (Invitrogen, Life Technologies, Carlsbad, Calif.) and treated with RNase-free DNase (Qiagen Inc., Valenica, Calif.). cDNA from human mRNA was prepared with 1 μg total RNA extracted with Omniscript® RT Kit (Qiagen Inc., Valenica, Calif.). Equal amounts of cDNAs were used for FVIII PCR with HotStarTaq DNA Polymerase (Qiagen Inc., Valenica, Calif.): 95° C. for initial denaturation followed by 35 cycles at 94° C.×30 seconds, 50° C.×45 seconds, 72° C.×1 minute, and final extension at 72° C.×7 min. FVIII primers were: 5′ccctattataagagcagaagttga3′ (SEQ ID NO:5) (forward); 5′ccaattaatcccgagtgcatatc3′ (SEQ ID NO:6) (reverse) with an expected fragment of 400 bp as previously described (39). From isolated liver cells, total RNA was extracted and first round of PCR was performed with primers described above followed by nested PCR on this product with the primers, 5′-cccatatataagagcagaagttga-3′ (SEQ ID NO:7) (forward) and 5′-gtttcttctaggttcttctcctc-3′ (SEQ ID NO:8) (reverse); the expected product was 133 bp (39). For mouse β-actin cDNAs were amplified with Platinum PCR Supermix (Invitrogen, Life Technologies, Carlsbad, Calif.) as follows: 94° C. for 3 minutes, 25 cycles at 94° C. for 30 seconds, 56° C. for 1 minute, 72° C. for 1 minute, and final elongation at 72° C. for 7 minutes. Primers were 5′-gtggggcgccccaggcacca-3′ (SEQ ID NO:9) (forward) and 5′-cttccttattgtcacgcacgatttc-3′ (SEQ ID NO:10) (reverse). PCR products were resolved in 1% agarose gel, the expected product was 540 bp (6).

cDNAs were prepared from 1 μg RNA extracted by Omniscript® RT Kit (Qiagen Inc., Valenica, Calif.) from total human BM and stromal, myeloid (CD33+), CD133+, and CD105+ BM cell fractions (STEMCELL Technologies, Vancouver, BC, Canada). For FVIII, equal amounts of cDNAs were subjected to PCR with Hot Start Taq DNA Polymerase (Qiagen Inc., Valencia, Calif.): 95° C. for initial denaturation, followed by 35 cycles at 94° C.×30 seconds, 52° C.×45 seconds, and 72° C.×1 minute, and final extension at 72° C.×7 minutes. FVIII primers were, 5′ttcttcagggaacagagggag3′ (SEQ ID NO:11) (forward) and 5′gagttatttcccgttgatgg3′ (SEQ ID NO:12) (reverse). For human β-actin, cDNAs were amplified with Platinum PCR Supermix (Invitrogen, Life Technologies, Carlsbad, Calif.) as follows: 94° C. for 3 minutes, 25 cycles at 94° C. for 30 seconds, 52° C. for 30 seconds, 72° C. for 1 minute, and final elongation at 72° C. for 7 minute. Primers were 5′agagctatgagctgcctgac3′ (SEQ ID NO:13) (forward) and 5′gtgatccacatctgctggaa3′ (SEQ ID NO:14) (reverse). PCR products were resolved in 1% agarose gel with the expected product was 400 bp.

FVIII immunostaining. To identify FVIII-expressing cells, LSEC and Kupffer cells were isolated by collagenase liver perfusion from C57/BL6 mice and hemophilia A mice and separated by immunomagnetic beads for CD45 and LSEC (Miltenyi Biotec, Auburn, Calif.). Cells were cultured for 48 hours on collagen-coated cover slips in M199 (LSEC) and IMDM (Kupffer cells) with 10% FBS and antibiotics, fixed in 4% PFA. BM-MSC were cultured on cover slips for 2 days and fixed in 4% PFA. Cells were stained with rabbit anti-FVIII antibody (1:100, Abcam, Cambridge, Mass.) for 1 hour at RT. After PBS washing, Alexa Fluor®488-conjugated goat anti-rabbit IgG (1:500, Molecular Probes, Life Technologies, Carlsbad, Calif.) was added for 1 hour. Nuclei were stained with DAPI-Antifade (Molecular Probes, Life Technologies, Carlsbad, Calif.).

FVIII western blots. Liver tissue or cell extracts were lysed in sample buffer containing a cocktail of protease inhibitors (Sigma, St. Louis, Mo.). Protein concentration was determined using BCA™ protein assay (Pierce, Thermo Fisher Scientific, Rockford, Ill.). Equal amounts of protein (50 g) were loaded per lane and separated by 6% SDS-PAGE. Proteins were transferred to polyvinylidene difluoride membrane (Biorad, Hercules, Calif.). FVIII was detected with antibody against FVIII (Abcam, Cambridge, Mass.) diluted 1:200. The blot was developed with ECL+ reagent (Amersham Biosciences, GE Healthcare, Piscataway, N.J.). Protein loading was normalized with actin as control.

Fluorogenic thrombin generation test (FTGT). Plasma was collected by tail-clip bleeds in 0.38% sodium citrate (wt/vol) and thrombin generation was assayed as previously described (6).

FVIII inhibitor assay. Bethesda assay (40) with Nijmegen modification (41) was used as previously described (6).

FVIII antigen immunoassay. 96-well microtitre polystyrene plates (Maxisorp, Nunc) were coated with 100 μl plasma (1:40 dilutions) in doubling dilutions in GBS buffer for overnight incubation at 4° C. After washing with PBS-Tween® (PBST) and saturation (1 hour at RT) with 200 μl blocking buffer (PBS-HSA 5%), 100 μl of rabbit polyclonal FVIII antibody specific for mouse FVIII (ab53703, Abcam, Cambridge, Mass.) was added (dilution 1:5000) in Tris-Casein buffer and plates were incubated at 37° C. for 2 hours. After washing with PBST, 100 μl alkaline phosphatase-conjugated goat polyclonal antibody against rabbit IgG (1:1000, ab6722-Abcam) in Tris buffer was added per well and incubated at 37° C. for 1 hour. After washing, 200 μl of SIGMA FAST p-nitrophenyl phosphate was added (Sigma) and incubated at RT in dark for 30-40 minutes. The reaction was stopped with 50 μl 3M NaOH and plates were read at 405 nm with SpectraMax™ reader (Molecular Devices, Sunnyvale, Calif.). Plasma from healthy C57BL/6 mice was analyzed concurrently.

Bleeding assay. Tail-clip challenge was performed to assess correction of the bleeding phenotype in hemophilia as previously described (5).

Statistical methods. Data are shown as means±SD. Significances were analyzed by t-test, Chi-square test or ANOVA, as applicable, with SigmaStat software (Jandel Scientific, San Rafael, Calif.). P<0.05 was considered significant.

### 2. Results

Bone marrow of hemophilia A mice was efficiently replaced by healthy donor BM with generation of rare endothelial cells or hepatocytes

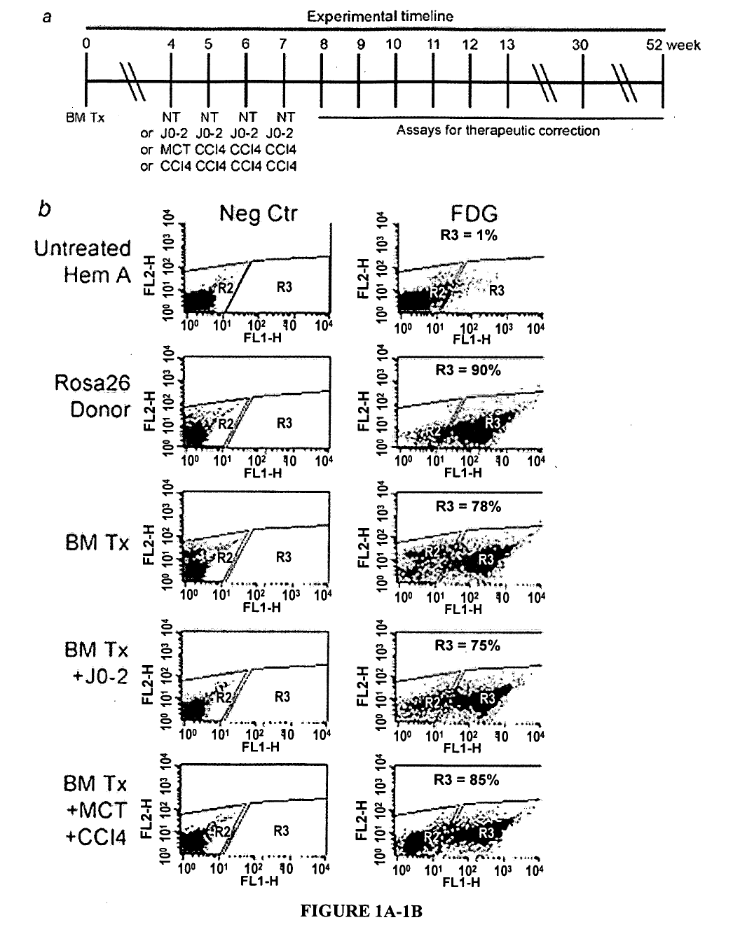

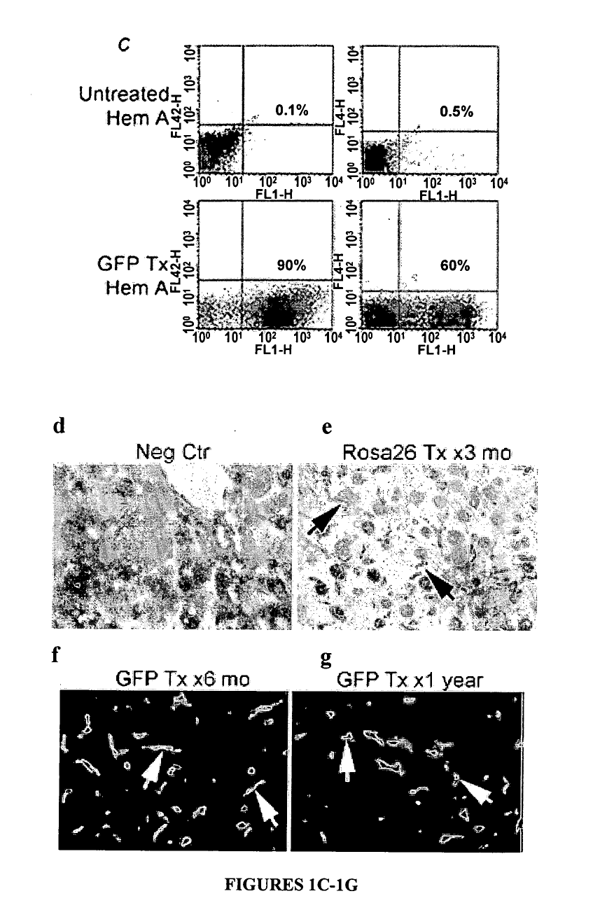

BM was transplanted in hemophilia A mice (FIG. 1A). To replace BM highly efficiently, hemophilia A mice were subjected to lethal total body radiation before transplanting total nucleated donor BM cells intravenously. To determine whether the number of donor BM-derived cells was relevant, 2×10⁶ or 10×10⁶ donor BM cells were transplanted in animal groups. To track transplanted cells, BM donors were Rosa26 β-galactosidase (LacZ) transgenic mice, green fluorescent protein (GFP)-transgenic mice, or CD45.1 donor mice, and to avoid rejection, hemophilia A mice were in syngeneic C57BL/6 background. To promote transdifferentiation of donor BM-derived cells, liver damage was induced in some mice 4 weeks after BM transplants with 4 weekly doses of 125 μg/kg J0-2 antibody, a ligand of apoptogenic Fas, which causes extensive hepatocyte and endothelial injury, or of 200 mg/kg monocrotaline (MCT), an agent that impairs survival and proliferation of LSEC and endothelial cells elsewhere, along with several doses of 0.5-1.0 ml/kg carbon tetrachloride (CCl₄), a lipid peroxidative toxin, which produces extensive hepatic, pulmonary and other tissue injuries (16, 17). Tissue histology 1-3 days after administration of these agents showed significant liver injury. The extent of BM chimerism was demonstrated 8 to 12 weeks after BM transplants, along with tail-clip challenge and other assays at intervals to demonstrate therapeutic correction of hemophilia A during 1 year. (Table 1)

TABLE 1
Flow cytometry in recipients of CD45.1 BM cells showing over
80% chimerism. These data were from a total of 7 mice.

| | Blood | BM |
|---|---|---|
| CD45.1 | 96 ± 2 | 93 ± 2 |
| Donor | | |
| CD45.1 | 86 ± 7 | 82 ± 7 |
| Hem A Tx | | |

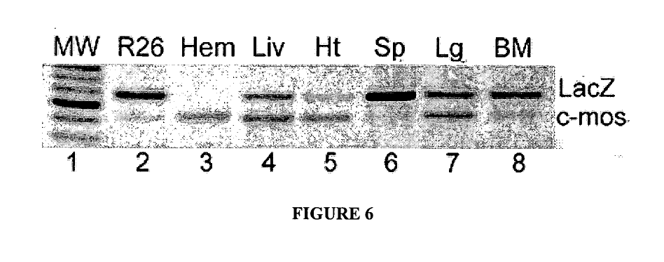

Flow cytometry for BM chimerism in LacZ, GFP or CD45.1 BM cell recipients indicated up to 90% of nucleated blood and BM cells were donor BM-derived (FIGS. 1B-1D). Genomic DNA PCR showed transplanted BM-derived cells in multiple organs, including liver, heart, lungs, and spleen, as expected (FIG. 6). By tissue staining, colonization of organs by transplanted cells was verified. For instance, the liver of recipient hemophilia A mice contained LacZ and GFP-positive donor BM-derived cells (FIG. 1D-1G). BM-derived cells were mostly in vascular spaces and were present in large numbers. This was maintained throughout the duration of the studies, including in mice with BM transplantation alone and mice additionally given J0-2, MCT and/or CCl₄.

Donor BM-derived hepatocytes were observed extremely rare and no donor BM-derived LSEC were found. To confirm absence of donor BM-derived endothelial cells, BM from Tie2-GFP transgenic mouse donors was transplanted, where GFP expression is restricted under control of the Tie2 promoter to only endothelial cells (6), into lethally-irradiated syngeneic FVB/N mice. These FVB/N recipients were treated with J0-2, MCT and CCl₄ after BM transplants, according to the above protocols (n=36). It was verified that BM was extensively replaced in these animals. Genomic DNA PCR for GFP transgene showed transplanted cells in multiple organs, which was similar to hemophilia A mice. Tissue surveys from FVB/N mice 3, 6 and 8 months after BM transplants in all conditions studied, including after organ injuries, showed no GFP-stained endothelial cells in the liver, BM, kidneys, spleen or heart, verifying that transplanted BM did not generate endothelial cells.

In further studies, BM from GFP-transgenic mice was transplanted into C57BL/6 mice with lethal total body irradiation, and 2 months later, administered the hepatotoxin, acetaminophen. Generation of donor BM-derived hepatocytes was analyzed after another 3 months. Despite extensive BM replacement, donor BM-derived hepatocytes were rarely observed by GFP staining of liver sections and flow cytometric analysis of liver cells isolated by collagenase digestion showed these constituted<0.2% of total hepatocytes. FVIII deficiency was corrected by BM transplantation and was related to the number of BM cells transplanted

118 hemophilia A mice receiving BM-transplants were grouped for studies of therapeutic correction (Table 2). Due to their delicate nature, some mice were lost through technical issues or complications of total body radiation, particularly in MCT or CCl₄-treated mice, where radiation increased hepatotoxicity. Although 4 doses of 1 ml/kg CCl₄ alone at 10 day intervals were planned, all mice in this group died after 3 doses of CCl₄, and this manipulation was not pursued further.

TABLE 2
Summary of mice in various treatment
groups after BM transplantation

| | Number of | Number of | |
|---|---|---|---|
| Animal groups | survivors after | evaluable mice | Therapeutic |
| with BM | BM Tx and | with FVIII | correction |
| transplantation | conditioning | (>5%) | (%) |
| No further | 42 | 23 | 55% |
| treatment | | | |
| JO-2 | 55 | 26 | 47% |
| CCl4 alone | 0 | — | — |
| MCT + CCl4 | 21 | 9 | 43% |
| All combined | 118 | 58 | 49% |

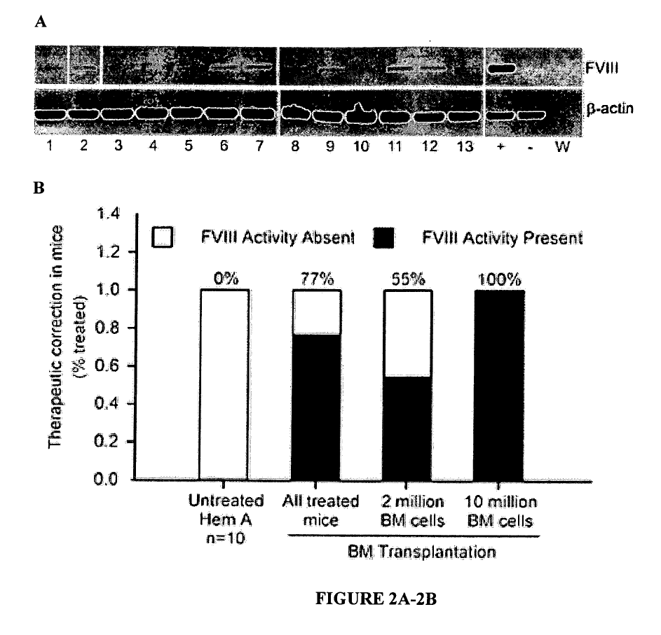

Therapeutic correction was defined as when plasma factor VIII activity levels exceeded 5% of normal, as indicated by ultrasensitive fluorimetric assay (6). Overall, 58 of 118 hemophilia A mice (49%) showed therapeutic correction after BM transplantation. In mice treated with 2×10⁶ BM cells plus either J0-2 (n=55) or MCT (n=21), plasma FVIII activity levels were similar to mice treated with 2×10⁶ BM cells alone, p=n.s. Therefore, hepatic or endothelial injury with J0-2 and MCT was not beneficial, which re-verified that these manipulations did not help generation of hepatic or endothelial cells by donor BM-derived cells. BM analysis indicated that FVIII mRNA was expressed in mice examined after BM transplantation and therapeutic correction (FIG. 2A). Next, it was analyzed whether the number of BM cells transplanted affected outcomes in hemophilia A mice. Although BM chimerism was achieved after transplantation of even 2×10⁶ donor BM cells, presence of additional cell types in BM itself could have produced such differences. In 86 hemophilia A mice subjected to BM transplantation, lesser therapeutic correction was found in recipients of 2×10⁶ BM cells compared with 10×10⁶ BM cells (55%, n=42, versus 100%, n=44, respectively, p<0.0001, Fisher's exact test) (FIG. 2B).

As BM was replaced extensively after transplantation of 2×10⁶, as well as 10×10⁶ BM cells, this remarkable difference in therapeutic efficacy required more analysis. A series of 30 hemophilia A mice treated with GFP transgenic BM alone were compared, including 18 with 2×10⁶ BM cells and 12 with 10×10⁶ BM cells. Plasma FVIII activity levels were analyzed from these mice 6 months after BM transplantation. Again, therapeutic correction was inferior in recipients of 2×10⁶ BM cells compared with 10×10⁶ BM cells, 61% versus 100%, p=0.02, Fisher's exact test.

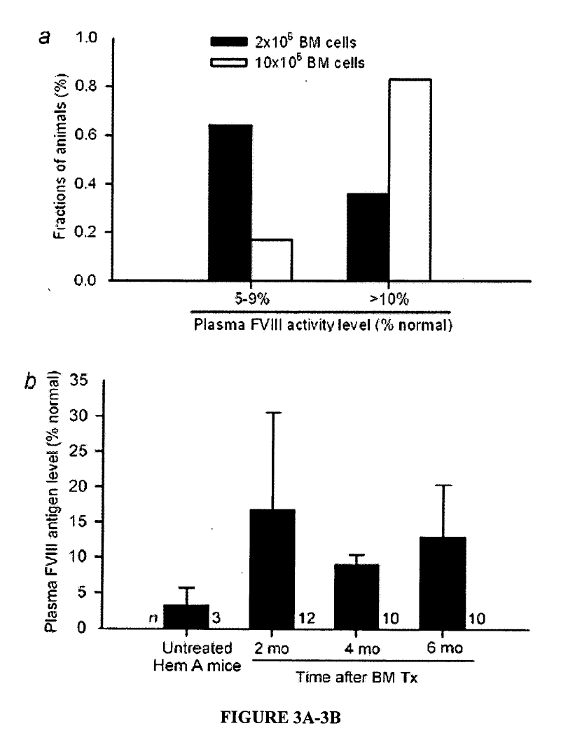

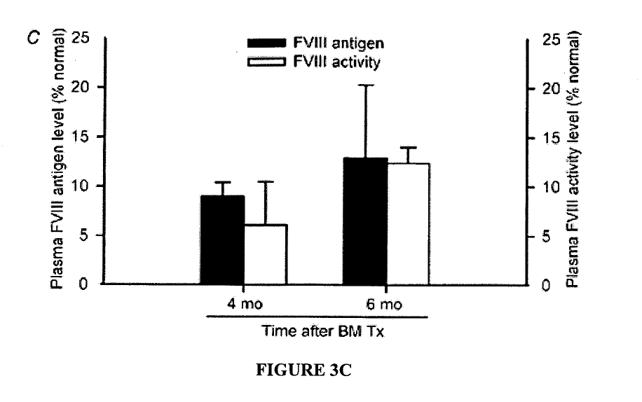

Plasma FVIII activity was also lower, 5 to 9%, in 7 of 11 mice (64%) given 2×10⁶ BM cells, and higher, >10%, in 10 of 12 mice (83%) given 10×10⁶ BM cells, p=0.036, Fisher's exact test (FIG. 3A). This further confirmed that transplantation of more BM cells was most effective, despite presence of adequate numbers of BM-repopulating cells after transplantation of either 2×10⁶ or 10×10⁶ BM cells. To evaluate whether plasma FVIII activity appropriately reflected FVIII protein synthesis and release, plasma FVIII antigen in several mice was measured 2, 4 and 6 months after transplantation of 10×10⁶ BM cells (FIG. 3B). Plasma FVIII antigen and activity levels were found to be in agreement with one another (FIG. 3C), increasing confidence in the findings. As FVIII antigen levels were maintained in a steady range without significant changes over many months after BM transplantation, it appears that FVIII-producing cells appeared early and their numbers remained stable after BM transplantation.

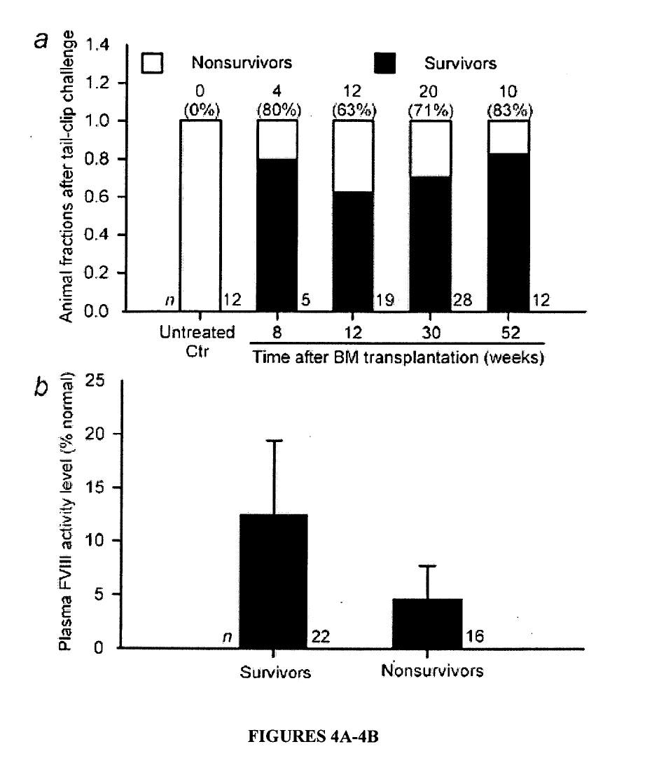

To investigate whether restoration of plasma FVIII activity protected mice from bleeding, tail-clip assays were performed in randomly selected mice 2, 3, 7 or 12 months after BM transplants. Survival in mice treated with healthy BM cells was 63%-83% compared with 0% in untreated control hemophilia A mice (FIG. 4A), p<0.05, ANOVA. This was in agreement with the assignment of therapeutic outcomes in mice treated with BM transplantation alone (Table 2). Plasma FVIII activity was significantly higher in mice surviving tail-clip challenge compared with nonsurvivors, 12.4±7.0% versus 4.6±3.1%, p<0.001, t-test (FIG. 4B).

Neutralizing antibodies against FVIII were not detected in 15 hemophilia A mice after BM transplants, including mice with or without detectable FVIII antigen and plasma FVIII activity (Table 3). It was noted that recipients were lethally-irradiated, BM chimerism was near-total, and hemophilia mice had not previously been sensitized to FVIII protein.

TABLE 3
Presence of plasma VIII activity and FVIII
antigen and antibody against FVIII

| | | Plasma | Plasma | Plasma FVIII |
|---|---|---|---|---|
| | | FVIII | FVIII | antibody |
| Experimental | Mouse | activity | antigen | (Bethesda |
| mice | ID | (% normal) | (% normal) | Units) |
| BM23 | 194 | 9.3 | Absent | Absent |
| 4 mo Post Tx | | | | |
| BM23 | 248 | 12.3 | 26.4 | Absent |
| 1 year Post Tx | | | | |
| BM17 | 226 | 22.9 | 6.0 | Absent |
| 1 year Post Tx | | | | |
| BM17 | 232 | 19.3 | 22.1 | Absent |
| 1 year Post Tx | | | | |

Nature of cell types responsible for therapeutic benefits after BM transplantation

To identify what specific cell types derived from donor BM were of therapeutic significance, tissues from multiple hemophilia A mice were surveyed, 4 to 6 months after transplanting GFP transgenic BM cells. Donor-derived GFP-positive cells were found in all organs, including BM, liver, spleen, lungs, heart, and kidneys (FIG. 1). In each case, donor BM-derived cells were in vascular spaces, and not in parenchyma, similar to the liver. In view of the major role of LSEC in producing FVIII, and previous reports by other groups of hepatic replacement by donor BM-derived cells (18, 19), focus was put on the liver. Only rare GFP-positive hepatocytes of donor BM origin were found, including in mice given J0-2 or MCT plus CCl₄, and these did not form cell clusters, which excluded transplanted cell proliferation. This was consistent with the absence of hepatocyte selection under these conditions, which should have been necessary for proliferation of donor BM-derived hepatocytes (16, 19). By contrast, characterization of GFP+ cells in vascular spaces showed abundant CD11b+Kupffer cells or mononuclear cells. Despite extensive and diligent searches for LSEC by costaining for GFP and CD31, an endothelial marker, donor BM-derived LSEC were not found in the liver, or other organs, e.g., spleen.

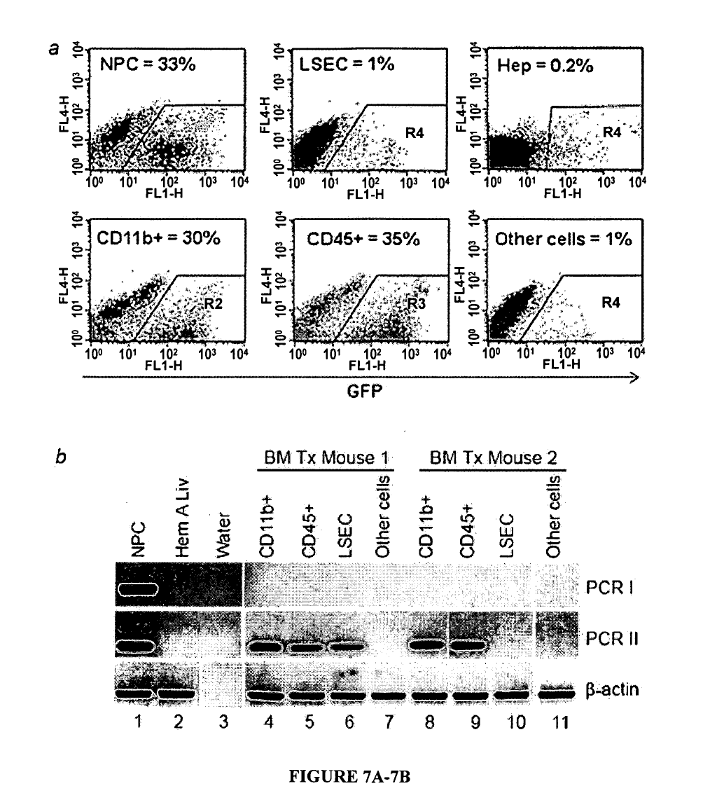

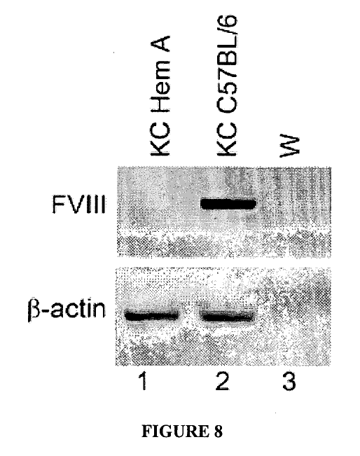

To identify FVIII production in donor BM-derived cell types, parenchymal and nonparenchymal cell subsets were isolated by collagenase perfusion of the liver from hemophilia A mice 3 to 6 months after transplanting GFP transgenic BM cells. BM-derived CD11b+ Kupffer cells or mononuclear cells were most abundant (FIG. 7). After further cell separations, FVIII mRNA expression in cell fractions was analyzed. This required nested RT-PCR, indicating low abundance of FVIII mRNA in donor-derived cells, which was different from the requirement of only one round of PCR for FVIII after RT of mRNA in healthy LSEC. However, isolation of CD11b+ cells followed by culture to eliminate potential contaminants, showed Kupffer cells expressed FVIII mRNA at greater levels, requiring only one round of PCR after RT (FIG. 8). Moreover, immunostaining with an antibody recognizing FVIII antigen in the immunoassay, verified expression of FVIII protein in CD45+ liver cells. FVIII protein in isolated cells could not be demonstrated by western blot of total cell lysates, indicating low levels of expression.

To conclusively verify whether FVIII expression in BM-derived mononuclear cells was sufficient for therapeutic benefits in hemophilia, Kupffer cells were isolated from healthy C57BL/6 mice by immunomagnetic sorting for CD11b. Cell phenotype was verified by flow cytometry for F4/80 antigen and macrophage function by assays of phagocytosis. 2×10⁶ Kupffer cells were then injected intravenously via tail vein into hemophilia A mice pre-treated by GdCl₃ to injure native mononuclear/macrophage cells. After 3 days, several of these mice (n=3), along with control untreated hemophilia A mice (n=3), were subjected to tail-clip challenge. All recipients of healthy Kupffer cells survived bleeding challenge, whereas none of the control mice survived. This proved that BM-derived mononuclear cells possessed the capacity to correct hemophilia A.

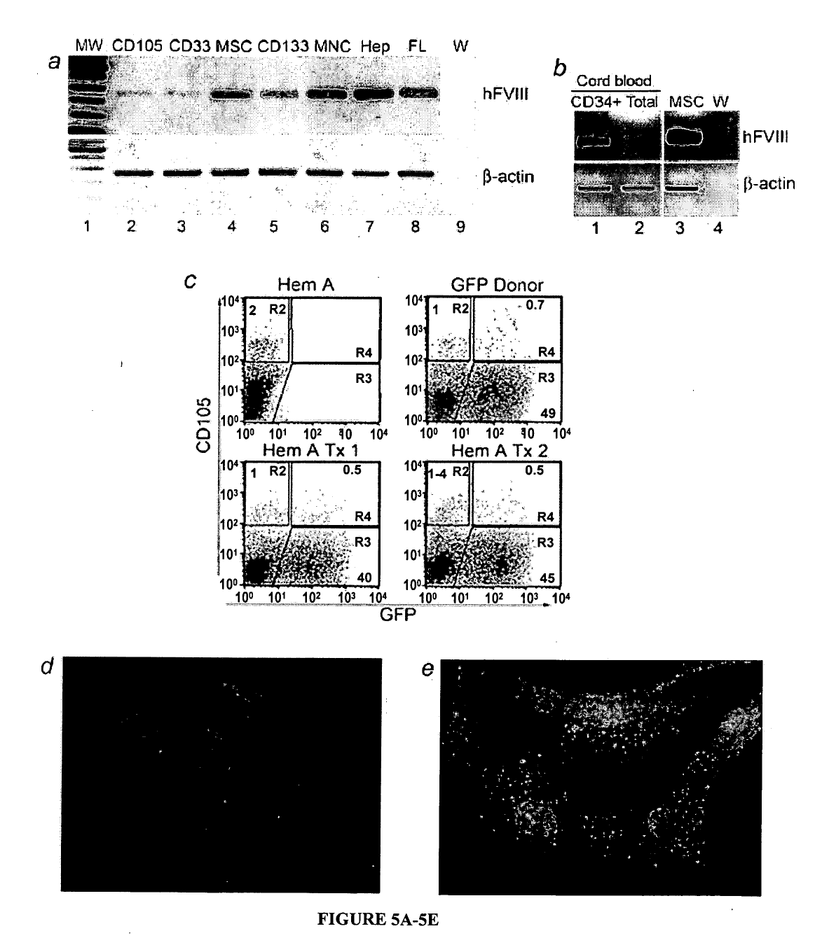

As total mouse BM showed FVIII mRNA expression, the fractions of BM cells contributing to FVIII synthesis was examined. Human BM was studied, with RNAs obtained from total nucleated human BM cells, as well as fractions with CD133 (hematopoietic progenitor) cells, CD33 (myeloid) cells, CD105 (marker of mesenchymal plus other lineages) cells, and mesenchymal stromal cells (hMSC). FVIII mRNA was found in various fractions of BM cells, including hMSC and CD133 cells (FIG. 5A). FVIII mRNA was also found in human cord blood (CB) cells (FIG. 5B). This led to an examination of whether BM transplantation in mice had generated mesenchymal cells. Donor BM-derived CD105+ cells were found in the BM of recipient mice (FIG. 5C). To determine whether BM-derived MSC were capable of expressing and synthesizing FVIII, human BM-derived cultured hMSC were studied and found by immunostaining to also be expressing FVIII protein (FIG. 5D-5E). To establish their therapeutic potential, hMSC were expanded in culture conditions and transplanted in NOD/SCID hemophilia A mice 5×10⁶ hMSC intraperitoneally, along with Cytodex3 microcarriers for extracellular support, according to published protocols (5). After tail-clip challenge 3 days later, in recipients of hMSC, bleeding ceased and 3 of 3 mice survived, which was in contrast with continued bleeding and mortality in untreated hemophilia A mice. This confirmed that BM-derived hMSC produced FVIII and could correct the bleeding phenotype of hemophilia A mice.

To determine whether transplantation of human BM or CB cells would have corrected FVIII deficiency, hemophilia A mice were studied in NOD/SCID background. Mice were sublethally irradiated to 350 Gy before transplantation of total human BM (n 10; 15×10⁶ cells) or human CB (n 10; 20×10⁶ cells). However, BM chimerism was found to be limited, at best, to <2% after 3 months, which was insufficient. Transplantation of 1×10⁵ CD34-positive human BM cells (n 8), including with 4×10⁵ hMSC (3 of 8 mice) did not improve BM chimerism either. This prevented further studies of human cells in the NOD/SCID hemophilia A mice.

### 3. Discussion

These studies established that BM-derived mononuclear cells and MSC can produce FVIII. This identification of relevant cell types, other than LSEC or extrahepatic endothelial cells, is of considerable therapeutic significance in the treatment of hemophilia A and advances concepts in sites of FVIII synthesis and release, which will help define pathobiological mechanisms in hemophilia.

Unfractionated BM cells were transplanted to include studies of cells other than BM-repopulating cells in FVIII replacement. Extensive BM chimerism was successfully achieved after transplantation of 2×10⁶ or 10×10⁶ nucleated BM cells, along with extensive replacement of mononuclear cells, including Kupffer cells, by donor BM-derived cells. These studies proved that donor BM-derived cells synthesized and released FVIII, as shown by the presence of FVIII mRNA, as well as protein, in Kupffer cells, and correction of bleeding phenotype in hemophilia mice after transplantation of healthy Kupffer cells. The short-term therapeutic bioassay of bleeding phenotype in hemophilia A mice had previously been validated with studies of candidate cell types (5). In particular, the successful correction of bleeding phenotype in hemophilia A mice after transplantation of only 2×10⁶ healthy Kupffer cells, which represented approximately 20% of the total mass of Kupffer cells, is highly significant, because BM transplantation would have reconstituted greater fractions of Kupffer cells, besides replacing significant additional numbers of mononuclear cells throughout the body. This overall magnitude of mononuclear cell and Kupffer cell replacement should have produced significant levels of plasma FVIII activity, as shown in the studies. Of course, the lower level of FVIII mRNA expression and abundance of FVIII protein in mononuclear cells, relative to LSEC, should indicate that LSEC and other endothelial cells are more prolific sources of FVIII.

As the extent of BM chimerism was similar after transplantation of either 2×10⁶ or 10×10⁶ BM cells, superior correction of hemophilia in the latter situation suggested that another BM-derived cell type, i.e., MSC, contributed in FVIII production. Several lines of evidences were gathered in support of MSC in this setting, including presence of FVIII mRNA in MSC-containing fraction of human BM, expression of FVIII protein in BM-derived hMSC, as well as correction of bleeding phenotype in hemophilia A mice after transplantation of hMSC.

By contrast, the BM transplantation studies substantiated that endothelial cells, including LSEC, are not donor BM-derived. Tissue analysis, as well as analysis of isolated cells from hemophilia mice after BM transplants, including induced injuries with MCT, J0-2 and CCl₄ to promote recruitment of BM-derived cells in organs, excluded that donor BM produced endothelial cells. In particular, absence of Tie2-GFP- or CD31-positive donor BM-derived cells in organs, including liver, spleen and lungs, supported this conclusion, which had previously also been made by others (20-22). Similarly, it was found that parenchymal cells, e.g., hepatocytes, were not derived to any significant extent from donor BM, excluding the possibility that these cells contributed in replacement of plasma FVIII activity, especially to the high levels observed in the studies.

The ability of endothelial cells in the liver and elsewhere, as well as of other nonhepatic cell types to synthesize and release FVIII, provides appropriate frameworks for defining alterations in plasma FVIII activity levels during liver injury or disease (23, 24). Transplantation studies showing the efficacy of dissociated spleen cells or of intact spleen tissue in people with hemophilia A should most likely be explained by FVIII synthesis and release in monocytes, besides endothelial cells, in these materials (7, 25, 26, 27). It should be noteworthy that spleen is a particularly prominent reservoir of mononuclear cells and macrophages (27). Moreover, in studies of splenic cell transplants, peak plasma FVIII activity levels were rapidly detected, within 4-7 days (25), which was in agreement with the studies of Kupffer cell transplantation, as well as hMSC transplantation, where the bleeding phenotype of hemophilia A mice was corrected within 3 days, revalidating the appropriateness of the short-term cell transplantation assay. Maintenance of plasma FVIII activity levels within a steady range after BM transplantation in the studies indicate that cells expressing FVIII were stably and indefinitely reconstituted. By contrast, although transplantation of splenic cells provided prolonged therapeutic benefits, ranging from 22 to 58 weeks, after BM transplantation, hemophilia A mice are considered to have been permanently cured.

Furthermore, this offers the basis of why plasma FVIII activity levels were preserved in a recent case, where OLT was performed in an individual from a donor with hemophilia (9). In this situation, FVIII production in extrahepatic cell populations should have been unperturbed, as was observed, and this was similar in some aspects to the experiment in hemophilia A mice treated with healthy donor BM. No systematic study of BM transplantation in people with hemophilia A has been undertaken. Although BM transplantation was studied in a canine model of hemophilia A some 40 years ago (28, 29), and BM transplantation in one child with hemophilia and aplastic anemia had been reported (30), their results will now need revisiting.

From a biological perspective, in view of the critical requirement of FVIII for blood clotting, it is possible to view maintenance of plasma FVIII activity at required levels through high capacity “central” systems, e.g., LSEC or endothelia in major organs would seem efficient for that purpose, with availability of lower capacity “auxiliary” systems in cell types capable of escaping injury or diseases afflicting endothelia, e.g., MSC within BM or elsewhere. Expression of FVIII in mononuclear cells may serve as yet another “peripheral” system to rapidly deliver FVIII in sites of tissue injury and active bleeding. This model should be useful for reconciling and addressing plasma FVIII levels in disease-specific mechanisms.

The identification of additional nonendothelial cell types capable of supporting FVIII synthesis and release provides targets for cell/gene therapy, including donor-derived stem cells from BM, mobilized peripheral blood, or CB (31, 32-34). Of course, BM-derived MSC will be relevant (35), as these may be readily expanded and genetically-modified (36, 37).

### REFERENCES

1. Bolton-Maggs, P. H. & Pasi, K. J. Haemophilias A and B. Lancet 361, 1801-1809 (2003).
2. Terraube, V., O'Donnell, J. S. & Jenkins, P. V. Factor VIII and von Willebrand factor interaction: biological, clinical and therapeutic importance. Hemophilia (2009).
3. Hollestelle, M. J., et al. Tissue distribution of factor VIII gene expression in vivo—a closer look. Thromb Haemost 86, 855-861 (2001).
4. Lewis, J. H., Bontempo, F. A., Spero, J. A., Ragni, M. V. & Starzl, T. E. Liver transplantation in a hemophiliac. N Engl J Med 312, 1189-1190 (1985).
5. Kumaran, V., et al. Transplantation of endothelial cells corrects the phenotype in hemophilia A mice. J Thromb Haemost 3, 2022-2031 (2005).
6. Follenzi, A., et al. Transplanted endothelial cells repopulate the liver endothelium and correct the phenotype of hemophilia A mice. J Clin Invest 118, 935-945 (2008).
7. Aronovich, A., et al. Correction of hemophilia as a proof of concept for treatment of monogenic diseases by fetal spleen transplantation. Proc Natl Acad Sci USA 103, 19075-19080 (2006).
8. Jacquemin, M., et al. FVIII production by human lung microvascular endothelial cells. Blood 108, 515-517 (2006).
9. Madeira, C. L., Layman, M. E., de Vera, R. E., Fontes, P. A. & Ragni, M. V. Extrahepatic factor VIII production in transplant recipient of hemophilia donor liver. Blood 113, 5364-5365 (2009).
10. Wion, K. L., Kelly D, Summerfield, J. A., Tuddenham, E. G. D., & Lawn, R. M. Distribution of factor VIII mRNA and antigen in human liver and other tissues. Nature 317, 726-729 (1985).
11. Lin, Y., et al. Use of blood outgrowth endothelial cells for gene therapy for hemophilia A. Blood 99, 457-462 (2002).
12. Bailey, A. S., et al. Transplanted adult hematopoietic stems cells differentiate into functional endothelial cells. Blood 103, 13-19 (2004).
13. Lancrin, C., et al. The haemangioblast generates haematopoietic cells through a haemogenic endothelium stage. Nature 457, 892-895 (2009).
14. Bi, L., et al. Targeted disruption of the mouse factor VIII gene produces a model of haemophilia A. Nat Genet. 10, 119-121 (1995).
15. Sarkar, R., et al. Total correction of hemophilia A mice with canine FVIII using an AAV 8 serotype. Blood 103, 1253-1260 (2004).
16. Mallet, V. O., et al. Bone marrow transplantation in mice leads to a minor population of hepatocytes that can be selectively amplified in vivo. Hepatology 35, 799-804 (2002).
17. Joseph, B., et al. Monocrotaline promotes transplanted cell engraftment and advances liver repopulation in rats via liver conditioning. Hepatology 44, 1411-1420 (2006).
18. Wang, X., et al. The origin and liver repopulating capacity of murine oval cells. Proc Natl Acad Sci USA 100 Suppl 1, 11881-11888 (2003).
19. Wang, X., et al. Cell fusion is the principal source of bone-marrow-derived hepatocytes. Nature 422, 897-901 (2003).
20. De Palma, M., Venneri, M. A., Roca, C. & Naldini, L. Targeting exogenous genes to tumor angiogenesis by transplantation of genetically modified hematopoietic stem cells. Nat Med 9, 789-795 (2003).
21. Perry, T. E., et al. Bone marrow-derived cells do not repair endothelium in a mouse model of chronic endothelial cell dysfunction. Cardiovasc Res (2009).
22. Yoder, M. C. & Ingram, D. A. The definition of EPCs and other bone marrow cells contributing to neoangiogenesis and tumor growth: Is there common ground for understanding the roles of numerous marrow-derived cells in the neoangiogenic process?Biochim Biophys Acta (2009).
23. Doering, C. B., Josephson, C. D., Craddock, H. N. & Lollar, P. Factor VIII expression in azoxymethane-induced murine fulminant hepatic failure. Blood 100, 143-147 (2002).
24. Hollestelle, M. J., Geertzen, H. G., Straatsburg, I. H., van Gulik, T. M. & van Mourik, J. A. Factor VIII expression in liver disease. Thromb Haemost 91, 267-275 (2004).
25. Liu, L., Xia, S. & Seifert, J. Transplantation of spleen cells in patients with hemophilia A. A report of 20 cases. Transpl Int 7, 201-206 (1994).
26. Jiang, H. C., et al. Ten-year experience with living related donated splenic transplantation for the treatment of hemophilia A. Transplant Proc 38, 1483-1490 (2006).
27. Swirski, F. K., et al. Identification of splenic reservoir monocytes and their deployment to inflammatory sites. Science 325, 612-616 (2009).
28. Marchioro, T. L., Hougie, C., Ragde, H., Epstein, R. B. & Thomas, E. D. Hemophilia: role of organ homografts. Science 163, 188-190 (1969).
29. Storb, R., et al. Canine hemophilia and hemopoietic grafting. Blood 40, 234-238 (1972).
30. Ostronoff, M., et al. Allogeneic bone marrow transplantation in a child with severe aplastic anemia and hemophilia A. Bone Marrow Transplant 37, 627-628 (2006).
31. Moayeri, M., Hawley, T. S. & Hawley, R. G. Correction of murine hemophilia A by hematopoietic stem cell gene therapy. Mol Ther 12, 1034-1042 (2005).
32. Kootstra, N. A., Matsumura, R. & Verma, I. M. Efficient production of human FVIII in hemophilic mice using lentiviral vectors. Mol Ther 7, 623-631 (2003).
33. Doering, C. B., Gangadharan, B., Dukart, H. Z. & Spencer, H. T. Hematopoietic stem cells encoding porcine factor VIII induce pro-coagulant activity in hemophilia A mice with pre-existing factor VIII immunity. Mol Ther 15, 1093-1099 (2007).
34. Gangadharan, B., Parker, E. T., Ide, L. M., Spencer, H. T. & Doering, C. B. High-level expression of porcine factor VIII from genetically modified bone marrow-derived stem cells. Blood 107, 3859-3864 (2006).
35. Pittenger, M. F., et al. Multilineage potential of adult human mesenchymal stem cells. Science 284, 143-147 (1999).
36. Van Damme, A., et al. Efficient lentiviral transduction and improved engraftment of human bone marrow mesenchymal cells. Stem Cells 24, 896-907 (2006).
37. Doering, C. B. Retroviral modification of mesenchymal stem cells for gene therapy of hemophilia. Methods Mol Biol 433, 203-212 (2008).
38. Benten, D., et al. Hepatic targeting of transplanted liver sinusoidal endothelial cells in intact mice. Hepatology 42, 140-148 (2005).
39. Chao, H., et al. Phenotype correction of hemophilia A mice by spliceosome-mediated RNA trans-splicing. Nat Med 9, 1015-1019 (2003).
40. Kasper, C. K., et al. Proceedings: A more uniform measurement of factor VIII inhibitors. Thromb Diath Haemorrh 34, 612 (1975).
41. Verbruggen, B., et al. The Nijmegen modification of the Bethesda assay for factor VIII:C inhibitors: improved specificity and reliability. Thromb Haemost 73, 247-251 (1995).

1. A method of treating a subject with hemophilia, the method comprising introducing into the subject a therapeutically effective amount of Factor VIII-producing cells so as to thereby treat the subject with hemophilia.

2. The method of claim 1, wherein the Factor VIII-producing cells are (i) bone marrow cells or (ii) a bone marrow cell fraction, obtained from a donor subject.

3. The method of claim 1, wherein the Factor VIII-producing cells are introduced into the subject with hemophilia by transplantation.

4. The method of claim 1, wherein the Factor VIII-producing cells are cord blood cells, or a cord blood cell fraction, or mobilized peripheral blood cells.

5. The method of claim 4, wherein the cord blood is introduced intravenously into the subject with hemophilia.

6. The method of claim 1, wherein the Factor VIII-producing cells comprise CD34+ cells.

7. The method of claim 1, wherein the Factor VIII-producing cells comprise CD133+ cells.

8. The method of claim 1, wherein the Factor VIII-producing cells comprise mononuclear cells.

9. (canceled)

10. (canceled)

11. (canceled)

12. The method of claim 1, wherein the mononuclear cells or mesenchymal stromal cells are extracted from bone marrow of the donor subject.

13. The method of claim 1, wherein the mononuclear cells or mesenchymal stromal cells are extracted from peripheral blood of the donor subject.

14. The method of claim 12, wherein the mononuclear cells are expanded in vivo before extraction.

15. The method of claim 12, wherein the mesenchymal stromal cells are cultured in vitro after extraction from the donor subject.

16. (canceled)

17. (canceled)

18. (canceled)

19. (canceled)

20. The method of claim 4, wherein the donor subject and the subject with hemophilia are the same subject in that the cord blood was previously obtained from the umbilical cord of the subject when the subject was neonatal.

21. The method of claim 1, wherein the Factor VIII-producing cells are introduced parenterally.

22. The method of claim 1, wherein the Factor VIII-producing cells are introduced by transfusion.

23. (canceled)

24. The method of claim 3, wherein the cells are transplanted into the cavity of a bone of the subject with hemophilia.

25. The method of claim 1, where the subject with hemophilia is a subject with hemophilia A.

26. A composition for treatment of hemophilia comprising an amount of Factor VIII-producing mononuclear cells or Factor VIII-producing mesenchymal stromal cells and a pharmaceutically acceptable carrier.

27. A composition for treatment of hemophilia comprising an amount of (a) (i) Factor VIII-producing bone marrow or Factor VIII-producing bone marrow fraction, or (ii) Factor VIII-producing cord blood cells or Factor VIII-producing cord blood fraction; or (i) and (ii); and (b) a pharmaceutically acceptable carrier.

28-31. (canceled)
